(19) United States
(12) Patent Application Publication
(10) Pub. No.: US 2025/0012791 A1
(43) Pub. Date: Jan. 9, 2025

(54) INDIUM TIN OXIDE FILM COATED UPSTANDING SILICON NANOWIRES (ITO/USNWs) AND THE USE THEREOF IN SENSITIVE NANOELECTRONIC DEVICE (NED)

(71) Applicant: National Chung Hsing University, Taichung City (TW)

(72) Inventors: Shu-Ping Lin, Taichung City (TW); Man-Cheng Sun, Taichung City (TW); Lester Uy Vinzons, Taichung City (TW); Yu-Ting Wei, Taichung City (TW)

(21) Appl. No.: 18/348,245

(22) Filed: Jul. 6, 2023

(51) Int. Cl. G01N 33/543; C23C 14/02; C23C 14/08; C23C 14/22; C23C 14/54; C23C 14/58; G01N 33/58

(52) U.S. Cl. CPC G01N 33/5438; C23C 14/021; C23C 14/086; C23C 14/221; C23C 14/542; C23C 14/5806; G01N 33/587

(57) ABSTRACT

A nanostructural sensing substrate includes indium tin oxide (ITO) film coated upstanding silicon nanowires (ITO/USNWs). The ITO/USNWs are fabricated by coating an ITO film on USNWs, the density of which has been reduced using a facile Ag-assisted chemical etching method. Furthermore, the bioreceptor modified ITO/USNWs are developed to serve as the sensing substrate of the EGFETs device for label-free diagnosis of biomarker related diseases, such as Alzheimer's disease (AD), acute myocardial infarction, coronary artery disease (CAD), hepatic encephalopathy, lung fibrosis, Cushing's syndrome and cancers. The ITO-coated-USNWs are also used in nano-featured cell based biosensors (CBB) for electrically quantitative evaluation of drug release.

### FIELD OF THE INVENTION

The present invention relates to a sensitive nanoelectronic device (NED) connected to an extended gate in the field-effect transistor (EGFET) with a nanostructural sensing substrate. Particularly, the present invention relates to a nanostructural sensing substrate comprising upstanding silicon nanowires coated with an indium tin oxide film (ITO/USNWs).

### BACKGROUND OF THE INVENTION

Extended-gate field-effect transistors (EGFETs) have been widely developed to address the problems encountered by using transistors for biological sensing, e.g., damage of conventional transistors from biomolecules, salts, and ions in the electrolyte.

Versatile structures and materials have been applied as sensing substrates of EGFETs, such as GaN nanowires (NWs), ZnO NWs, coaxial-structured ZnO/SiNWs, CuO NWs, ZnO/Ta thin films, PdO membrane, InZnxOy membrane, ZnO nano-array, InN/InGaN quantum dot, and indium tin oxide (ITO) thin film (Ahmed, N. M., et al., Semicond Sci Tech 36 (4), 2021). Among the various nanostructures available, upstanding NWs (USNWs) are particularly interesting owing to their ability to interact extensively with the nanoscale features of biomolecules. Notably, surface modification and functionalization of the sensing substrates improve the sensing sensitivity and specificity of EGFETs, thus expanding the applications of EGFETs to the sensing of various biological substances.

In this invention, it is aimed to develop a nanostructured sensing substrate that comprises upstanding silicon nanowires coated with an indium tin oxide film (named ITO/USNWs). The modified nano-coarse surface of the ITO coated upstanding nanowires can be used as a sensing substrate in EGFETs for the label-free and specific detection of certain biomarkers of a disease.

### SUMMARY OF THE INVENTION

Accordingly, in one aspect, the present invention relates a nanostructural sensing substrate comprising upstanding silicon nanowires (ITO/USNWs) coated with an indium tin oxide (ITO) film, wherein the upstanding silicon nanowires have an average height of 0.01-10 μm, and the ITO film has thickness of 10 nm-600 nm. Preferably, the upstanding silicon nanowires have an average height of 0.01-4 μm, and the ITO film has thickness of 100 nm-400 nm.

In certain embodiments of the invention, the ITO/USNWs comprise a nano-coarse surface with adequate surface roughness (Ra)>400 nm. Preferably, the Ra of the nano-coarse surface is in a range of 50-1000 nm. In a preferable embodiment of the invention, the Ra of the nano-coarse surface is in a range of 400-650 nm.

In certain embodiments of the invention, the ITO/USNWs are further coated with small molecules a small molecule containing silanol group, such as 3-aminopropyl trimethoxysilane (APTMS), on the nano-coarse surface for further conjugating with a cell, a protein or a bioreceptor. In certain embodiments of the invention, the bioreceptor includes, but is not limited to, an antibody of a biomarker. In one embodiment of the invention, the bioreceptor is cardiac troponin I antibody (cTnIAb). In other embodiment of the invention, the bioreceptor is neurofilament-L antibody (NFLAb). In one embodiment of the invention, the bioreceptor is L-lactate dehydrogenase (LDH). In one embodiment of the invention, the bioreceptor is cortisol antibody (AHC).

In another aspect, the present invention provides a method for preparing the said indium tin oxide coated upstanding silicon nanowires (ITO/USNWs), which comprises steps of: forming Ag nanoparticles (AgNPs) on a Si surface; Ag-assisted chemical etching in a solution of HF and H₂O₂ for 0.5-4 minutes to create a high-aspect-ratio nano-coarse surface of USNWs; reducing the density of USNWs using KOH/IPA; and ITO coating on USNWs to produce ITO/USNWs. The said ITO/USNWs are used to serve as a sensing substrate in EGFETs device.

In certain embodiments of the invention, the Ag-assisted chemical etching is conducted for 3-4 minutes in the dark. In certain embodiments of the invention, the density reducing step is conducted in an aqueous solution containing 4-4.45 M KOH and 0.8-2.61 M isopropanol (IPA) for 5 sec-2 min, preferably 20-30 seconds.

In certain embodiments of the invention, the preparing method of ITO/USNWs further comprises modification of ITO/USNWs surface with 3-aminopropyl trimethoxysilane (APTMS).

In another aspect, the present invention provides a method for label-free diagnosis of Alzheimer's disease (AD), comprising detecting NFL-specific neuron-derived exosomes (NDEs) in a liquid biopsy from a subject by using the nanostructural sensing substrate comprising indium tin oxide coated upstanding silicon nanowires (ITO/USNWs). In certain embodiments of the invention, the nano-coarse surface of ITO/USNWs are modified with 3-aminopropyl trimethoxysilane (APTMS) and neurofilament-L antibody (NFLAb). In a certain embodiment of the invention, the sensing substrate is connected to an extended gate in a field-effect transistor (EGFET).

In another aspect, the present invention provides a method for real-time diagnosis of acute myocardial infarction, comprising detecting human cardiac troponin I (cTnI) in a liquid biopsy from a subject by using the nanostructural sensing substrate comprising indium tin oxide coated upstanding silicon nanowires (ITO/USNWs). In certain embodiments of the invention, the nano-coarse surface of ITO/USNWs are modified with 3-aminopropyl trimethoxysilane (APTMS) and cTnI antibody (cTnIAb) to form cTnIAb-modified ITO/USNWs. In an embodiment of the invention, the nanostructural sensing substrate is integrated into a sensitive nanoelectronic device (NED) impedimetric system and served as a working electrode.

### BRIEF DESCRIPTION OF THE DRAWINGS

FIG. 1 is a schematic diagram of the nano-coarse surface of an exemplary sensing substrate prepared by a method of present invention.

FIGS. 2A-2C show Morphology of the nano-coarse surface of the sensing substrate determined by using SEM and AFM. SEM confirmation of the vertical alignment of USNWs after the MACE process (FIG. 2A), the USNWs (FIG. 2B), and the ITO/USNWs (FIG. 2C). Magnification: 1000 K× in FIG. 2B and FIG. 2C.

FIGS. 3 A-3D show the responses of the APTMS/ITO/USNWs EGFET in solutions with different pH values (pH=3-11). FIG. 3A shows the transfer characteristics (I_{D}-V_{G}) in different pH buffer solutions. FIG. 3B shows the threshold voltage in the solution (V_{T,sol}) and pH are linearly correlated. FIG. 3C shows the output characteristics (I_{D}-V_{D}) in the saturation region in different pH buffer solutions. FIG. 3D shows the plot of (I_{D})^{1/2} and pH in the saturation region are linearly correlated.

FIGS. 4A-4F show the detection of NFL-specific NDEs in clinical blood plasma. Transfer characteristics for cognitively healthy (CH) individuals (FIG. 4A), patients subjective cognitive decline (SCD) patients (FIG. 4B), mild cognitive impairment (MCI) patients (FIG. 4C), and dementia due to AD (DAD) patients (FIG. 4D). FIG. 4E shows the calibrated responses for all clinical subjects. FIG. 4F shows the statistical analyses of the four tested groups (n=13 in CH group; n=6 in SCD group; n=9 in MCI group; n=14 in DAD group); value (calibrated response)=mean±SD, ^{a}p<0.05, significant difference relative to CH. ^{b}p<0.05, significant difference relative to SCD. ^{c}p<0.05, significant difference relative to MCI.

FIGS. 5A-5D show the Impedance spectra of the (FIG. 5A) resistance, (FIG. 5B) reactance, and (FIG. 5C) calibration curve of different concentrations of cTnI. Error bars represent mean±SEM (n=6). FIG. 5D shows the demonstration of selectivity of cTnIAb-modified ITO-coated-USNWs using BSA.

FIG. 6A shows an exemplary nano-featured Cell based biosensor (CBB) for non-Faradaic EIS measurement. FIG. 6B shows distinct electrical signal changes in impedance spectra during the cell culture and the drug (125 ug/mL curcumin) treatment.

### DETAILED DESCRIPTION OF THE PREFERRED EMBODIMENTS

The present invention provides a nanostructural sensing substrate comprising indium tin oxide (ITO) coated upstanding silicon nanowires (ITO/USNWs). The USNWs are produced by metal-assisted chemical etching of silicon in HF/H₂O₂/H₂O solution using silver nano ions as catalyst, followed by etching in KOH/IPA solution to produce a suitable density of USNWs, and then ITO films are plated on upstanding silicon nanowires as a sensing conductive layer. The next step is to modify the surface of the ITO-coated USNWs with a self-assembled monolayer (SAM) of (3-aminopropyl)trimethoxysilane (APTMS) for the immobilization of bioreceptors, such as cardiac troponin I antibody (cTnIAb), neurofilament-L antibody (NFLAb), L-lactate dehydrogenase (LDH), cortisol antibody (AHC), and the like.

The other characteristics and advantages of the present invention will be further illustrated and described in the following examples. The examples described herein are intended for illustrations, not for limitations of the invention.

### Example 1. Preparation of Indium Tin Oxide (ITO) Film Coated Upstanding Silicon Nanowires (ITO/USNWs)

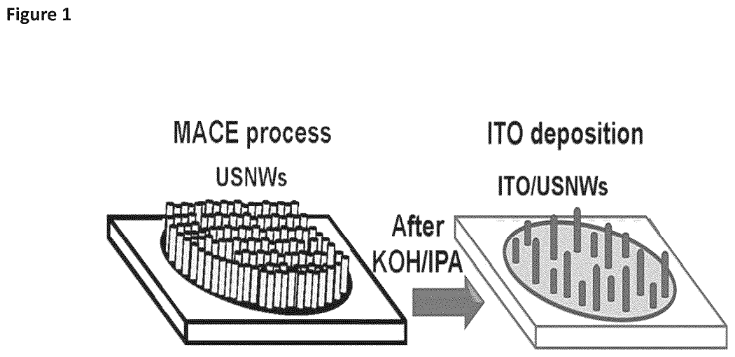

FIG. 1 is a schematic diagram of the preparation of a nano-coarse surface of USNWs in sensing substrate for further connecting to the gate of a metal-oxide-semiconductor field-effect transistor (MOSFET). After reducing the density of USNWs using KOH/IPA, ITO is coated on USNWs (ITO/USNWs) to serve as the sensing substrate of the EGFETs device. The preparing method is described as follow.

Firstly, High aspect-ratio nano-coarse surface of USNWs is fabricated using a facile metal-assisted chemical etching (MACE) method. Si wafers are cleaned by ultrasonication in ultrapure water, acetone, and 95% ethanol, and then dried with N₂. To prevent the unpolished back side of the Si wafers from being etched, it is spin-coated with AZ1518 photoresist at 2,000 rpm for 30 s and then baked at 100° C. for 1.5 min on a hotplate. The Si piece is immersed in 20 mL of 3% HF for 3 min to remove the native oxide layer, then immediately immersed in 20 mL of an aqueous solution of 0.005 M AgNO₃ and 4.8 M HF for 4 min in the dark for the electroless deposition of AgNPs. After gentle rinsing in ultrapure water, the Si piece is immersed in an etching solution containing 8.61 M HF, 0.45 M H₂O₂, and 48 M H₂O for 3 min in the dark to create upstanding silicon nanowires (USNWs).

After USNWs are formed, the AgNP catalysts are removed by thoroughly rinsing with ultrapure water and then immersed in 20 mL of 1:1 (v/v) aqueous solution of HNO₃ for 10 min. After thorough rinsing with ultrapure water, the Si sample is subjected to a 3-min oxide strip in 20 mL of 5% HF and then rinsed again with water. Finally, the photoresist coating on the back side of the Si sample is dissolved with acetone, and the sample is rinsed with 95% ethanol and dried on a hotplate at 110° C. To reduce the areal density of USNWs for conformal coating of ITO in the next step, USNWs are further etched in 20 mL of an aqueous solution containing 4.45 M KOH and 2.61 M isopropanol (IPA) for 20 s. The etched samples are then rinsed with 95% ethanol and dried on a hotplate at 110° C.

Conformal coating of ITO film is performed using an ion-assisted electron-beam deposition system (SGC-22SA; Showa Shinku Co., Ltd., Japan) with O2 (10 sccm) as the reactive gas and Ar (12 sccm) as the working gas. The chamber is pumped to 2×10⁻³ Pa before evaporation, and the pressure is kept at 5×10⁻² Pa during evaporation. The anode voltage and filament current are respectively set to 130 V and 2.3 A, resulting in a deposition velocity of 2.9±0.1 Å/s. Planar ITO films on glass slides are also prepared using the same process. The thickness (approximately 400±50 nm) of the deposited ITO film is determined using a stylus profilometer (Dektak; Bruker Corp., MA, USA). After coating with ITO, the samples are annealed in an oven at a temperature of 500° C. for 1 h to form a more compact ITO film with higher conductivity. ITO/USNWs (FIG. 1B) are then further cleaved into 1.1×1.3 cm² pieces, which are used as the sensing substrate of a EGFETs device.

The surface morphology of the sensing substrate is examined using field-emission scanning electron microscopy (FE-SEM; ZEISS Ultra Plus; ZEISS, Germany) and atomic force microscopy (AFM; DI-3100; Veeco, NY, USA). SEM images are obtained by FE-SEM in InLens mode with an accelerating voltage of 10 kV. AFM is performed in the soft tapping mode with a scan size of 10×10 μm² (for glass, USNWs, ITO/USNWs, and APTMS/ITO/USNWs) with a scan rate of 0.598 Hz. Each scan had 256 lines with 512 samples/line. The AFM data are visualized and analyzed using NanoScope Analysis (version 1.70; Bruker Corp., MA, USA) for the determination of surface average roughness (Ra).

The nano-coarse surface of USNWs was created after 20-s etching, and it showed a significantly rougher surface (657.33±46.10 nm) in comparison with ITO film (2.07±0.62 nm) and USNWs after the MACE process (123.33±17.12 nm). Although the Ra of ITO/USNWs decreased (568.17±46.48 nm), it still retained the fair roughness of the nano-coarse surface. The insignificant difference in Ra between USNWs and ITO/USNWs indicates the ITO film is conformally deposited on the nano-coarse surface of USNWs.

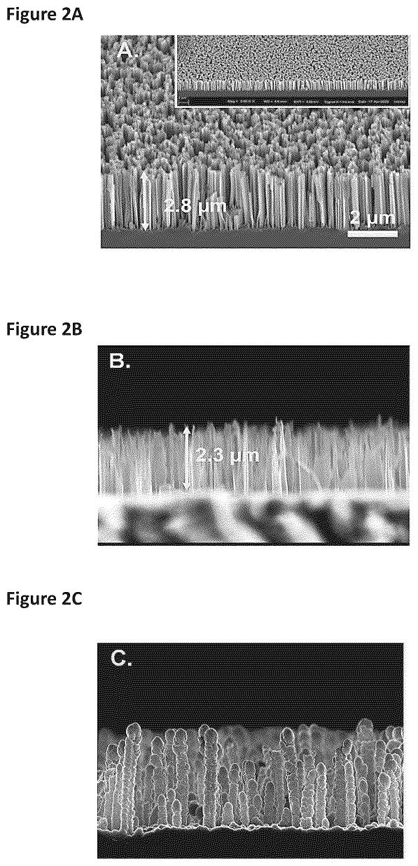

SEM images of USNWs fabricated on a silicon substrate via the MACE process show that USNWs are uniformly and vertically aligned on the surface of the substrate, with a height of approximately 2.8 μm and a high aspect ratio (FIG. 2A). After KOH/IPA etching for 20 s, the USNWs are noticeably changed with a decrease in their density and height (2.3 μm) (FIG. 2B). After the deposition of 400-nm ITO film on USNWs, the height of ITO/USNWs increased to about 2.8 μm in FIG. 2C. It is shown that a nano-coarse surface of nanostructured sensing substrate, USNWs with controlled areal density, can be successfully fabricated via MACE and KOH/IPA etching for conformal coating of an ITO and subsequent surface modifications.

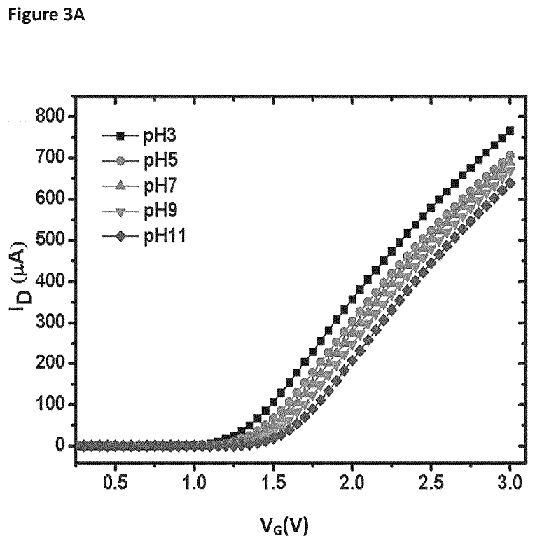

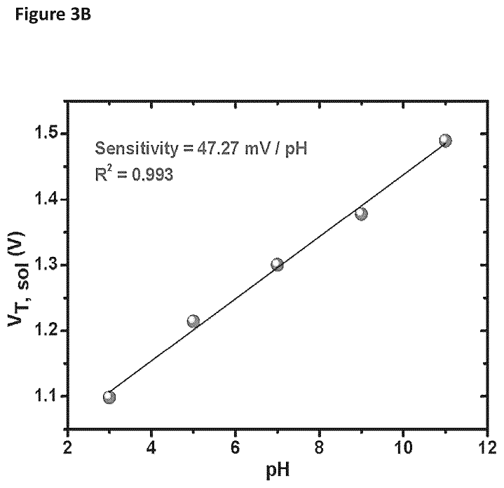

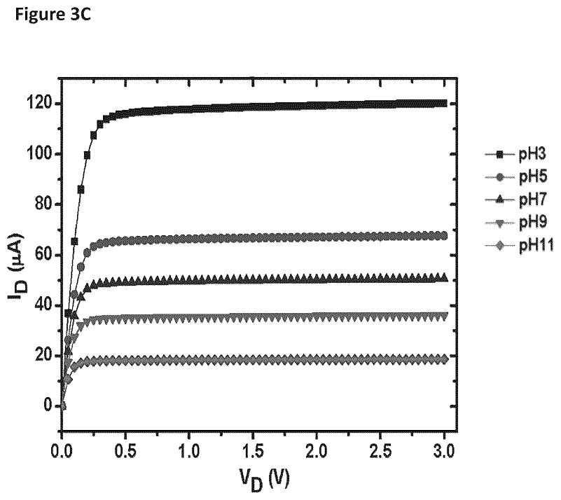

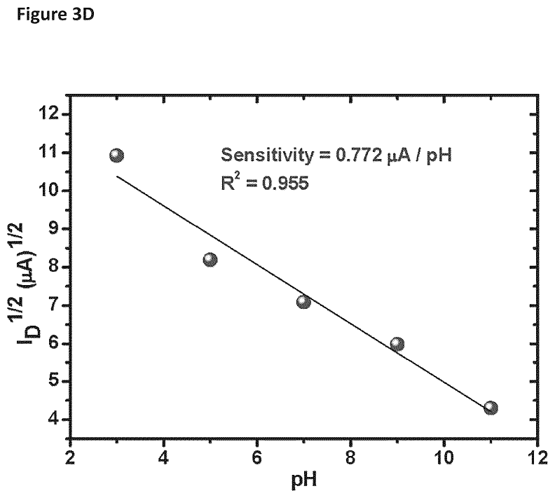

To quantify the performance of our as-fabricated APTMS/ITO/USNWs EGFET for further application to NDE sensing, the transfer characteristics operating in the linear region (I_{D}-V_{G} curves) and output characteristics in the saturation region (I_{D}-V_{D} curves) are obtained using solutions with different pH values (pH=3-11), as shown in FIGS. 3A to 3D. According to the MOSFET theory, the response of I_{D} in the linear and saturation regions are respectively expressed by equations (1) and (2):

$${I}_{D} = \frac{{μ}_{u {C}_{ox} W}}{2 L} [ 2 ( {V}_{G} - {V}_{T} ) {V}_{D} - {V}_{D}^{2} ] \qquad ( 1 )$$

$${I}_{D} = \frac{{μ}_{u {C}_{ox} W}}{2 L} [ {( {V}_{G} - {V}_{T} )}^{2} ] \qquad ( 2 )$$

Where μn denotes the carrier mobility of the channel, Cox indicates the gate oxide capacitance per unit area, and W and L stand for the width and length of the channel, respectively. V_{G}, V_{T}, and V_{D} represent the reference electrode voltage (V_{REF}), the threshold voltage, and the drain to source voltage of the EGFET sensor, respectively. When an EGFET sensor is immersed in an electrolyte solution, V_{T} dramatically changes because of a shift in the surface potential at the interface between the functionalized sensing surface, i.e., antibodies or chemical functional groups, and the electrolyte solution. Therefore, the threshold voltage in the solution (V_{T,sol}) can be expressed by equation (3) to further analyze signals using our APTMS/ITO/USNWs EGFETs:

$${V}_{T , sol} = {V}_{T} + {E}_{ref} - {Φ}_{0} + {χ}^{sol} - {φ}_{M} / q \qquad ( 3 )$$

Where E_{ref} is the reference electrode potential and equivalent to V_{REF}, Φ₀ is the surface potential at the interface between the functionalized surface of the sensing substrate and the electrolyte solution, χ^{sol} is the surface potential of the buffer solution, and φM is the work function of the conductive reference electrode. The I_{D}-V_{G} curves of APTMS/ITO/USNWs EGFET are measured by sweeping V_{G} from 0 to 3 V at constant V_{D}=0.3 V.

As shown in FIG. 3A, V_{T,sol} shifted from the left to the right as the pH increased, i.e., from high to low H⁺ ion concentrations, with an increase in the OH-concentration. This indicates that negative charges are attracted to the APTMS-modified sensing surface, and negatively charged ions (OH⁻) repel the electrons in the n-channel, thus decreasing current ID. According to the plot of V_{T,sol} as a function of pH in FIG. 3B, the as-fabricated APTMS/ITO/USNWs EGFET device is sensitive to changes in pH, with a high sensitivity response of 47.27 mV/pH and linearity of 0.99 in the pH range of 3 to 11.

FIG. 3C shows the I_{D}-V_{D} curves of APTMS/ITO/USNWs EGFET in the saturation region, which are measured by sweeping V_{D} from 0 to 3 V at constant V_{G}=1.5 V in solutions with different pH values (pH=3-11). According to equation (2), the response of the device to changes in pH can be evaluated by analyzing current (I_{D})^{1/2}. The decrease in current I_{D} in the n-channel is ascribed to negative hydroxyl ions in basic solutions. As shown in FIG. 3D, the plot of (I_{D})^{1/2} as a function of pH confirmed that the APTMS/ITO/USNWs EGFET had an excellent pH sensitivity of 0.77 (μA)^{1/2}/pH with a linearity of 0.96. The abovementioned electrical characteristics of the nanostructured sensing substrates of present invention indicate that the vertically upstanding nanostructure can significantly improve the sensing sensitivity and stability.

### Example 2. Detection of Brain-Derived Exosomes Using a Sensitive Nanoelectronic Device (NED) Containing Indium Tin Oxide (ITO)-Coated Upstanding Silicon Nanowires (ITO/USNWs)

NFL has been demonstrated as a promising biomarker in the serum and cerebrospinal fluid for the prediction of Alzheimer's disease (AD) progression. The ability of exosomes to readily cross the blood-brain barrier (BBB) is an important property that renders them particularly good biomarkers of CNS diseases and treatment response. Therefore, the detection of Neurofilament light chain (NFL)-specific NDEs provides information for the early diagnosis of dementia. In this example, a nanostructured sensing substrate is developed to capture NFL-specific NDEs in physiological samples for the label-free sensitive and specific distinction between normal people and varied patients with different stages associated with AD by EGFETs.

Firstly, the sensing substrate of Example 1 is functionalized with 3-aminopropyl trimethoxysilane (APTMS) for pH sensing, and then the NFL antibody (NFLAb) is immobilized on the APTMS for NFL-specific NDE detection. For Alzheimer's disease diagnosis, the Ra of the nano-coarse surface is preferably in a range of 400-650 nm. The transfer characteristics of the developed APTMS-ITO-coated USNWs (APTMS/ITO/USNWs) EGFET device in solutions with different pH values are verified for the device performance based on the sensing sensitivity and stability.

Subsequently, the performance of the NFLAb-modified APTMS/ITO/USNWs (NFLAb/APTMS/ITO/USNWs) EGFET device is assessed for the detection of clinical blood plasma. Clinical measurements were directly performed on native blood plasma samples, without needing any NFL-specific NDEs isolation in this example. Clinical blood plasmas are collected from 13 cognitively healthy (CH) individuals as well as 29 patients with subjective cognitive decline (SCD), mild cognitive impairment (MCI), and dementia due to AD (DAD). Electrical measurements were carried out using a semiconductor parameter analyzer (HP4145B) controlled by a computer via LabVIEW. The characteristic curves were obtained by sweeping the gate and drain voltages (V_{G} and V_{D}, respectively) with the source voltage held at 0 V and then measuring the resulting output drain current (I_{D}). Owing to device-to-device variation, the transconductance (gm=dI_{D}/dV_{G}) of the device has been used to calibrate the absolute change in current (|ΔI_{D}|) in clinical examinations. Similarly, the responses of our device are calibrated by dividing |ΔI_{D}| by the gm in phosphate-buffered saline (PBS) of the device and showed label-free significant differences in the plasma of a patient with dementia due to AD. Finally, the detection of NFL-specific NDEs using NFLAb/APTMS/ITO/USNWs EGFET is confirmed with electron spectroscopy for chemical analysis (ESCA), atomic force microscopy (AFM), and scanning electron microscopy (SEM).

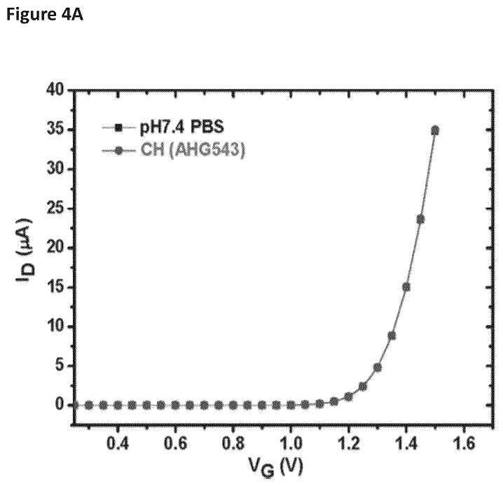

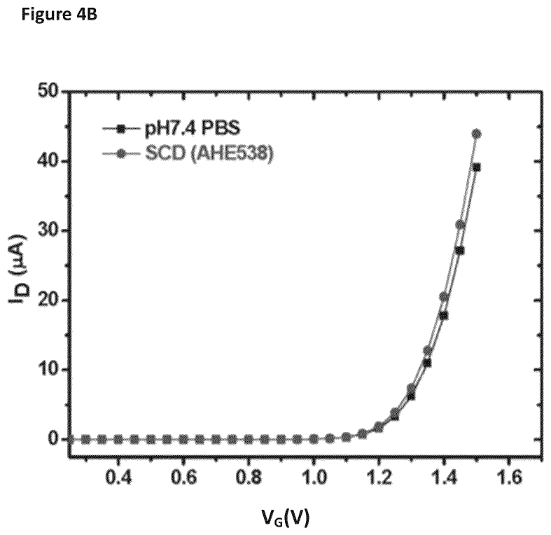

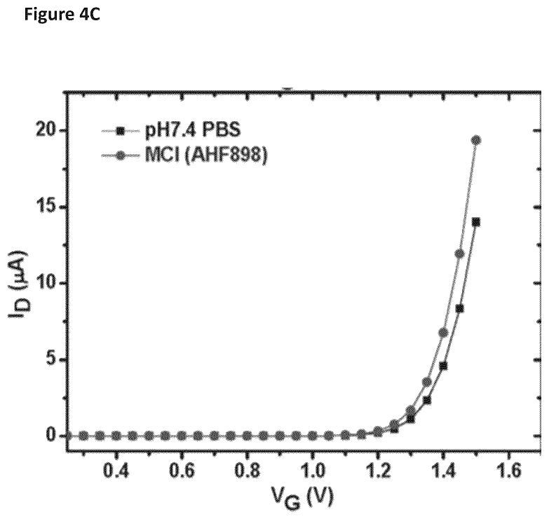

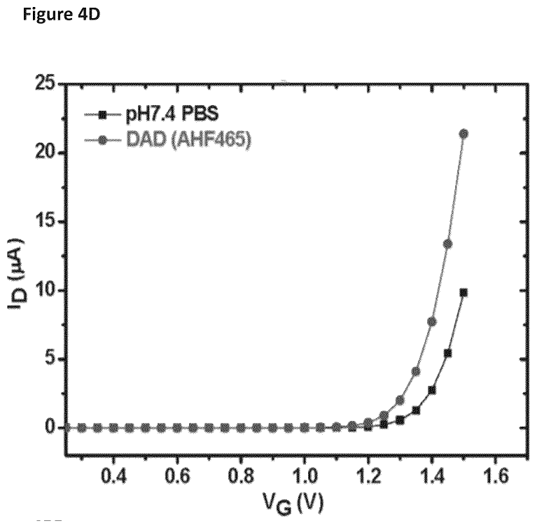

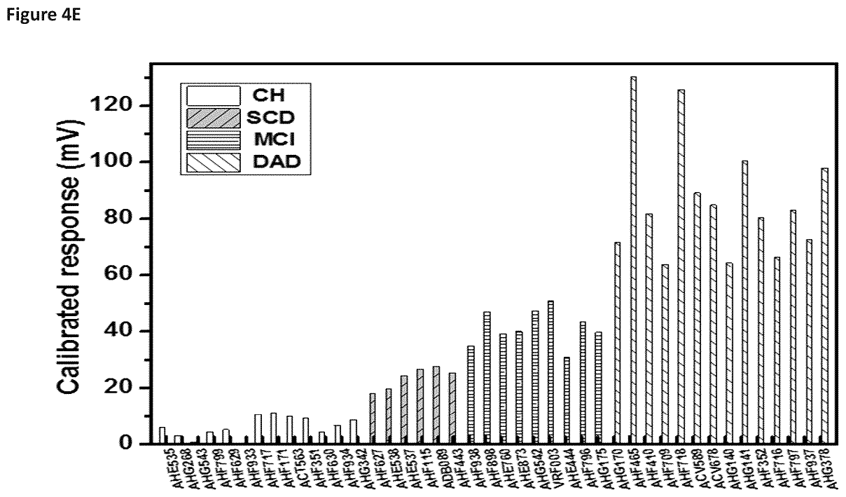

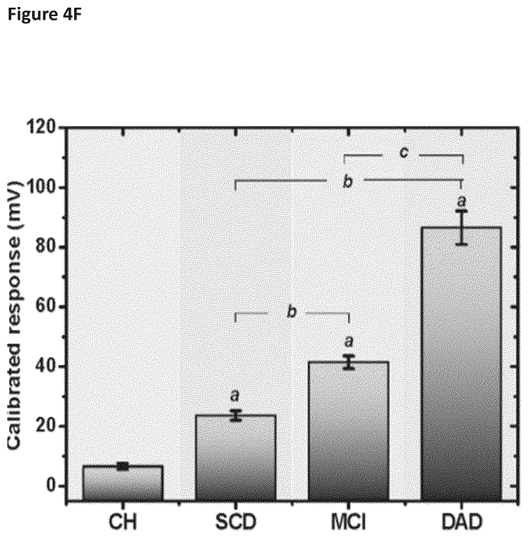

The apparent changes of |ΔI_{D,blood}| can be found at the beginning of the introduction of plasma samples to the NFLAb/APTMS/ITO/USNWs EGFETs. Representative transfer curves of NFLAb/APTMS/ITO/USNWs EGFETs for the CH, SCD, MCI, and DAD groups are displayed in FIGS. 4A-4D. The output currents differed between the cognitively impaired groups (29 patients) and the control group (13 healthy individuals). The results showed in FIGS. 4A-4D indicate that there are more NFL-specific NDEs in blood plasma from patients with more severe cognitive dysfunction, with distinct current changes on the developed NFLAb/APTMS/ITO/USNWs EGFETs. In order to suppress device-to-device variation in NFL specific NDEs measurement in blood plasma, the response is further calibrated by dividing |ΔI_{D,blood|} by the transconductance of the device performance in PBS (g_{m,PBS}=dI_{D,PBS}/dV_{G,PBS}), as expressed in the following equation.

$$\frac{\vert Δ {I}_{D , blood} \vert}{\frac{{dI}_{D , PBS}}{{dV}_{G , PBS}}} = \frac{\vert {I}_{human blood} - {I}_{PBS} \vert}{{g}_{m , PBS}} = Δ {V}_{Calibrated response} .$$

The calibrated response (ΔV) is no longer a function of the device performance, and it only depends on the absolute change in current induced by bound NFL-specific NDEs. Detailed calibrated responses for all clinical subjects are shown in FIG. 4E. As shown in FIG. 4F, the estimated average calibrated responses for SCD (23.64±1.57 mV, n=6), MCI (41.48±2.11 mV, n=9), and DAD (86.60±5.59 mV, n=14) are respectively 3.60, 6.32 and 13.20 times and significantly higher than that for CH (6.56±1.04 mV, n=13) (p<0.05). Moreover, the significant calibrated response for DAD suggests that the level of NFL-specific NDEs is higher in the DAD stage than in the other two prodromal stages (SCD and MCI) and healthy control in FIG. 4F.

Surface modification of the present developed nano-coarse surface of sensing substrate renders the NDE specific sensitive interface in the detection of NFL-specific NDEs in physiological plasma for the label-free fast distinction between normal people and varied patients with different stages associated with AD.

Other than neuroimaging recommended by most clinical guidelines, it is demonstrated that the developed nano-coarse surface leverages modified nanostructured sensing substrate on EGFET to detect NFL-specific NDEs with a LOD of 60 NDE/mL and LOQ of 6×10³ NDE/mL. A correlation between the level of NFL-specific NDEs in blood plasma and the severity of dementia, especially in DAD patients, is label-free fast discriminated within 2 min using NFLAb/APTMS/ITO/USNWs EGFETs. The captured NFL-specified NDEs on the nano-coarse surface of modified EGFETs can be further eluted and processed to extract vital dementia-related proteins in future.

### Example 3. Detection of Cardiac Troponin I (cTnI) Using a Sensitive Nanoelectronic Device (NED) Containing Indium Tin Oxide (ITO)-Coated Upstanding Silicon Nanowires (ITO/USNWs)

Currently, biomarkers of human cardiac troponin I (cTnI) and cTnT are considered the gold standard for the diagnosis of AMI incidence because of their high sensitivity. In this example, a sensitive nanoelectronic device (NED), containing indium tin oxide (ITO)-coated upstanding silicon nanowires (ITO/USNWs) as prepared in Example 1, is used to detect cTnI in the human blood sera. The morphology of ITO-coated-USNWs have a length of 2.5 μm. The EDX analysis shows 16.07% of the atomic content of indium of ITO. After cTnI antibody (cTnIAb) modification, various concentrations of cTnI in the human blood sera are then detected by cTnIAb-modified-ITO-coated-USNWs via microfluidic systems.

Polydimethylsiloxane (PDMS) microfluidic channels (16 mm×1 mm×0.06 mm) are fabricated via the standard microfabrication techniques and used to deliver sensing substances. After that, the PDMS microfluidic channel is assembled on the top of the exposed sensing area of the NED to enable the sample liquid flow over the analytical zone using a syringe pump. The Ag/AgCl reference electrode (RE) (ALS, RE-1S) is used to serve as a counter electrode on ground potential. The clinical experiments on the various concentrations of cTnI in the patient's blood sera are approved by Changhua Christian Hospital Ethics Committees (IRB No. 170113). The detection of known concentrations of cTnI in the human blood sera with a dilution factor of 10 is used to verify the sensing performance. Impedance measurements are performed using the E4980A precision LCR meter (Agilent Technologies) in frequency sweep mode and operated at 2 mV from 20 Hz to 2 MHz. Analytical results in terms of impedimetric constituents, resistance and reactance, are expressed. Statistical significance is determined with the unpaired t-test. The sensing selectivity is demonstrated by bovine serum albumin (BSA, 66.5 kDa).

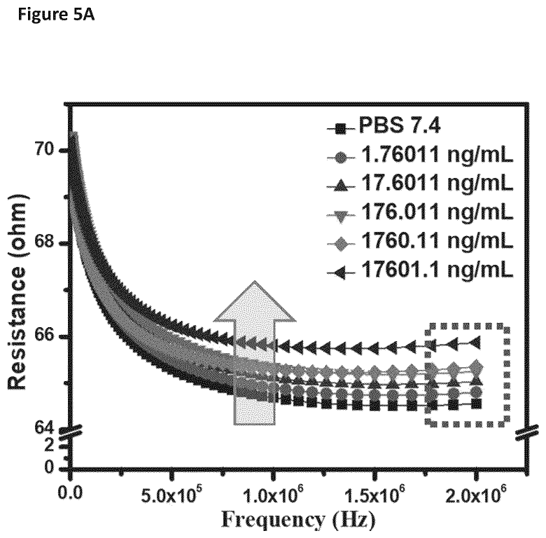

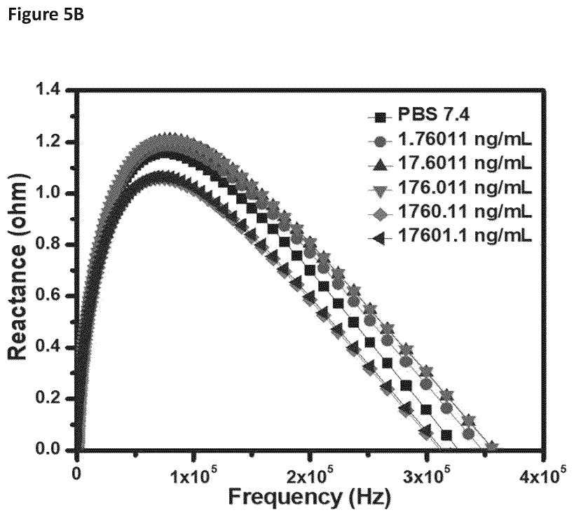

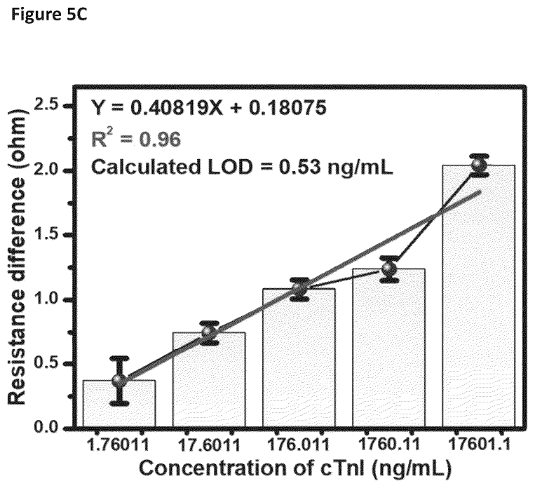

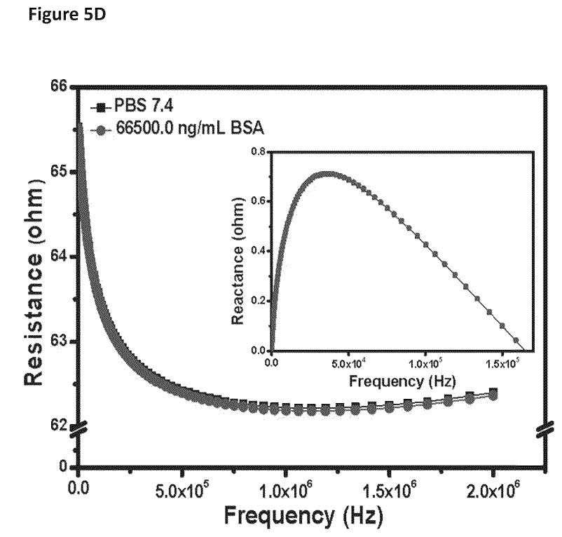

As shown in FIG. 5A, the changes in the resistance are proportional to the increased concentrations of cTnI. Reactance revealed a shift at a higher frequency owing to the capacitance effect of cTnI on cTnIAb-modified-ITO-coated-USNWs. FIGS. 5A and 5B show a direct correlation to the interfacial interaction between charged cTnI and its sensing surface. The changes in the resistance are calibrated as a function of different concentrations of cTnI in FIG. 5C. A regression equation (y=0.40819 x+0.18075) is then obtained in the range of 1.76011-17601.1 ng/mL. Based on our results, a limit of detection (LOD) for cTnI is calculated as 0.53 ng/mL (R²=0.96). In comparison with FIGS. 5A and 5B, no clear change in resistance (FIG. 5D) and reactance (an inset of FIG. 5D) in the detection of BSA (66500.0 ng/mL) shows better sensing selectivity. In light of the cTnI detection using impedance measurement, we summarized sensing features of two articles via varied nanomaterials to prove the feasibility of our work in Table 1. As a result, the cTnIAb-modified-ITO-coated-USNWs of present invention demonstrates the comparable sensing results in the detection of cTnI.

TABLE 1
Comparison table of cTnI detection via different nanomaterials.

| | Detection | Linear detection | |
|---|---|---|---|
| Electrode | techniques | range | Sensitivity/LoD |
| WO₃/ITO | Impedance | 1-250 | 26.56 Ω ng⁻¹ mL |
| | | ng mL⁻¹ | cm⁻²/16 ng mL⁻¹ |
| Carbon | Impedance | 5.0-100 | Increased sensitivity/ |
| nanofiber | | ng mL⁻¹ | 0.2 ng mL⁻¹ |
| cTnIAb- | Impedance | 1.76011-17601.1 | Good sensitivity |
| modified-ITO- | | ng mL⁻¹ | and selectivity/ |
| coated-USNWs | | | 0.53 ng mL⁻¹ |

### Example 4. Use of ITO-Coated-USNWs in Cell Based Biosensors (CBB) for Electrically Quantitative Evaluation of Drug Release

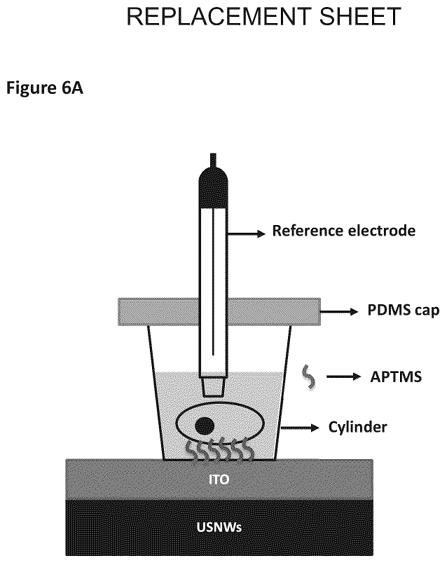

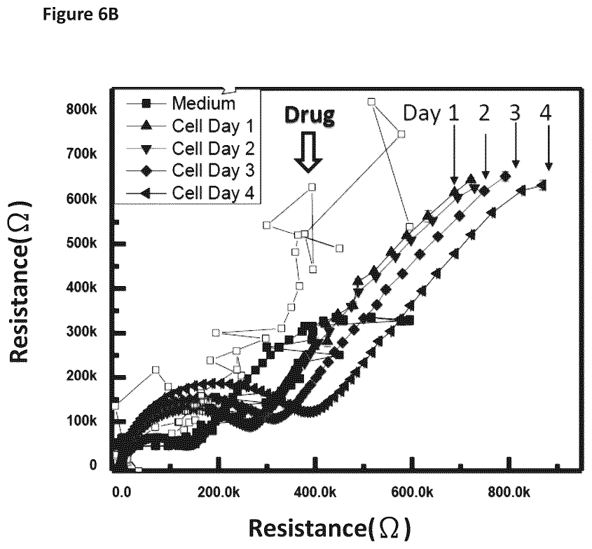

A nano-featured Cell based biosensor (CBB) is shown in FIG. 6A. Indium tin oxide (ITO)-coated upstanding silicon nanowires (ITO/USNWs) are prepared as described in Example 1. After 3-aminopropyl trimethoxysilane (APTMS) modification of the ITO/USNWs, a polystyrene cylinder with PDMS cap is attached on the top of device. MCF7-cells (human breast adenocarcinoma, ATCC HTB-22) were cultured at the density of 2.9×10⁴ cells/cm² in the DMEM with 10% FBS at 37° C. in 5% CO₂. For drug treatment, 125 μg/mL curcumin-encapsulated nanocarriers are loaded into the nano-featured CBB.

Keysight E4980A precision LCR meter is used in this Example. E4980A is operated at 1 mV with a frequency range of 20 Hz to 2 MHz with 100 sample points to record the reactance as a function of resistance for live-dead observation of MCF-7 cells. As shown in FIG. 6B, the nano-featured CBB of present invention will provide high stability and repeatability in various sensing environments. At day 1, day 2 and day 3 of the cell culture, the nano-featured Cell based biosensor shows distinct changes in impedance spectra. A disorder signal is observed after the treatment of curcumin-encapsulated nanocarriers.

Although a limited number of embodiments are described to illustrate the practice of the present invention, those skilled in the art may still make modifications or changes according to the description. Therefore, the scope of the present invention should only be limited by the claims of the patent, and not limited to the above examples.

What is claimed is:

1. A nanostructural sensing substrate, comprising upstanding silicon nanowires (ITO/USNWs) coated with a indium tin oxide (ITO) film, wherein the upstanding silicon nanowires have an average height of 0.01˜10 μm, and the ITO film has thickness of 10˜600 nm.

2. The nanostructural sensing substrate of claim 1, wherein the ITO/USNWs comprise a nano-coarse surface with an adequate surface roughness (Ra) of 50˜1000 nm.

3. The nanostructural sensing substrate of claim 2, wherein the Ra of the nano-coarse surface is in a range of 400˜650 nm.

4. The nanostructural sensing substrate of claim 1, wherein the nano-coarse surface of the ITO/USNWs are further modified with a small molecule containing silanol group, for further conjugating to a cell, a protein or a bioreceptor.

5. The nanostructural sensing substrate of claim 4, wherein the molecule containing silanol group is 3-aminopropyl trimethoxysilane (APTMS).

6. The nanostructural sensing substrate of claim 4, wherein the bioreceptor is an antibody.

7. The nanostructural sensing substrate of claim 6, wherein the bioreceptor is a neurofilament-L antibody (NFLAb).

8. The nanostructural sensing substrate of claim 4, wherein the bioreceptor is L-lactate dehydrogenase (LDH).

9. The nanostructural sensing substrate of claim 4, wherein the bioreceptor is a cortisol antibody (AHC).

10. A method for preparing the nanostructural sensing substrate of claim 1, comprising steps of:
forming Ag nanoparticles (AgNPs) on a Si surface;
Ag-assisted chemical etching in a solution of HF and H₂O₂ for 0.5˜4 minutes to create a highaspect-ratio nano-coarse surface of USNWs;
reducing the density of USNWs using KOH/isopropanol (IPA); and
ITO coating on the treated USNWs to produce ITO/USNWs in a sensing substrate.

11. The method of claim 10, wherein the step of Ag-assisted chemical etching is conducted for 3˜4 min in the dark.

12. The method of claim 10, wherein the step of density reducing is conducted in an aqueous solution containing 4˜4.45 M KOH and 0.8˜2.61 M isopropanol (IPA) for 5 seconds to 2 minutes.

13. The method of claim 10, wherein the method further comprises a step of modifying the ITO/USNWs surface with a small molecule containing silanol group.

14. A method for label-free diagnosis of Alzheimer's disease (AD), comprising detecting NFL-specific neuron-derived exosomes (NDEs) in a liquid biopsy from a subject by using the nanostructural sensing substrate of claim 1 comprising indium tin oxide coated upstanding silicon nanowires (ITO/USNWs).

15. The method of claim 14, wherein the nano-coarse surface of ITO/USNWs are modified with 3-aminopropyl trimethoxysilane (APTMS) and neurofilament-L antibody (NFLAb).

16. The method of claim 14, wherein the sensing substrate is connected to an extended gate of a metal-oxide-semiconductor field-effect transistor (MOSFET) in a field-effect transistor (EGFET) device.

17. The method of claim 14, wherein the nanostructural sensing substrate is integrated into a sensitive nanoelectronic device (NED) impedimetric system and served as a working electrode.
